(19) United States
(12) Patent Application Publication
(10) Pub. No.: US 2021/0304893 A1
(43) Pub. Date: Sep. 30, 2021

(54) Projection System for Visual Morphing of Patient Disease

(71) Applicant: University of South Carolina, Columbia, SC (US)

(72) Inventors: Richard Hoppmann, Columbia, SC (US); Keith Barron, JR., Columbia, SC (US); Robert Haddad, Columbia, SC (US); Steven Wilson, Columbia, SC (US); Floyd E. Bell, III, Lexington, SC (US)

(73) Assignee: University of South Carolina, Columbia, SC (US)

(21) Appl. No.: 17/183,663

(22) Filed: Feb. 24, 2021

Related U.S. Application Data
(60) Provisional application No. 62/982,158, filed on Feb. 27, 2020.

(51) Int. Cl. G16H 50/20; G16H 50/30; G16H 30/40; G06T 7/00

(52) U.S. Cl. CPC G16H 50/20; G06T 7/0016; G16H 30/40; G16H 50/30

(57) ABSTRACT

The subject matter disclosed herein is generally directed to methods and systems for generating, projecting, and displaying a patient's disease state based on the patient's medical images wherein the projected disease state can be compared to the patient's present state of health to provide data and material for patient education, healthcare provider education, clinical practice, and medical research.

### TECHNICAL FIELD

The subject matter disclosed herein is generally directed to methods and systems for generating, projecting, and displaying a patient's disease state based on the patient's medical images wherein the projected disease state can be compared to the patient's present state of health to provide data and material for patient education, healthcare provider education, clinical practice, and medical research.

### BACKGROUND

Many patients are unable to completely understand their health condition and any diseases they may have, how a disease can progress, and why they need to take medications and change certain life habits. This can lead to patients not being adequately motivated to take better care of themselves and negatively affect compliance with prescribed medical regimens.

Accordingly, it is an object of the present disclosure to present patients with a visual assessment of their illness and how it can progress if not treated effectively. This can both help educate patients about their disease, as well as motivate them to take better care of themselves by setting goals and being able to track improvements or setbacks in their disease. This will lead to more informed and better decision making, better healthcare outcomes, and decreased healthcare cost.

Visual assessment of a medical condition over time has not currently been effectively used in medicine. Visual assessment and outcome projections based on big data and deep learning will allow for more personalized and precise medicine to be practiced. Further, adding a visual component to learning disease pathology, pathophysiology, and clinical treatment can enhance learning of not only patients but also healthcare providers and those in training—a picture can truly be “worth a thousand words.” The system and its databases can also be used as research tools to assess effectiveness of specific treatment regimens and understanding of the disease process. Algorithm morphed images can also be used to expand or augment training datasets for AI development for a variety of visual dependent applications.

### SUMMARY

The above objectives are accomplished according to the present disclosure by providing a method for generating and projecting disease visual appearance. The method may include providing a morphing software platform, inputting at least one set of medical data into the morphing software platform, generating at least one visual interpretation of a change in medical status from the morphing platform based on the at least one set of medical data. Further, the method may include analyzing the medical data with an artificial intelligence platform to assist with generating the visual interpretation of the change in medical status. Still yet, the method may include projecting a disease visual appearance formed from the visual interpretation of the change in medical status. Again further, the method may be employed in an educational, clinical care, and/or research setting. Still further, the method may display the visual interpretation of the change in medical status in a patient-healthcare provider relationship. Yet more, the method may include assessing impact of a medical treatment via comparison of the visual interpretation of the change in medical status over a course of the medical treatment. Further still, the method may include augmenting at least one image dataset for artificial intelligence training based on analysis of the visual interpretation of the change in medical status. Still yet, the method may include designating at least one generated first visual interpretation of the change in medical status from a first image examination as a source image morphing input and designating at least one generated second visual interpretation of the change in medical status from a second image examination as a target image output and forming a morphed visual interpretation via comparing the first visual interpretation and the second visual interpretation to show changes in a medical condition. Still further, the at least one generated first visual interpretation may be taken earlier in time as compared to the at least one generated second visual interpretation. Furthermore, the at least one generated first visual interpretation may be taken later in time as compared to the at least one generated second visual interpretation. Still, the method may include generating a backward morphing of patient medical condition images via comparing the visual interpretation of the change in medical status from the morphing software platform based on the at least one set of medical data to at least one image where no medical condition is present. Moreover, the method may define a pathological processes via comparing at least two distinct visual interpretations of the change in medical status. Further yet, the method may include capturing patient disease morphing images and/or video. Yet still, the method may use at least one visual interpretation to assess treatment options for a medical condition. Further, the method may include assessing disease progress via analysis of the at least one visual interpretation. Still moreover, the method may include incorporating morphing algorithms and artificial intelligence into medical imaging devices for real-time assessment of a medical condition.

In an alternative embodiment, a system for projecting disease visual appearance is provided. The system may include an artificial intelligence platform, at least one set of medical data, a morphing software platform, and the artificial intelligence platform works in tandem with the morphing software platform to show a visual change in a medical status based on the at least one set of medical data. Further, a display may be provided for showing a disease visual appearance formed from the visual interpretation of the change in medical status. Still yet, the system may be employed in an educational, clinical care, and/or research setting. Yet moreover, the system may include a morphed visual interpretation created from at least one generated first visual interpretation of a first change in medical status from a first image examination as a source image morphing input and at least one generated second visual interpretation of a second change in medical status from a second image examination as a target image output to show changes in a medical condition.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present disclosure will be obtained by reference to the following detailed description that sets forth illustrative embodiments, in which the principles of the disclosure may be utilized, and the accompanying drawings of which:

FIG. 1 represents one embodiment of a projection system for Visual Morphing of Real Time or Recorded Patient Disease with patient's real time image of the heart on the left and the projected image on the right with increased thickness of the muscular wall (left ventricular hypertrophy or LVH) due to uncontrolled high blood pressure at one year.

FIG. 2 represents a system projection for a patient's echocardiogram at time zero to ventricular wall thickening and enlarged left atrium at one year and heart failure at five years.

FIG. 3 represents a method for applying morphing software to illustrate medical condition projections for medical disorders.

FIG. 4 represents a method for comparing a known database to specific patient data to form morphed visual projections for analysis.

The figures herein are for illustrative purposes only and are not necessarily drawn to scale.

### DETAILED DESCRIPTION OF A PREFERRED EMBODIMENT

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless specifically stated, terms and phrases used in this document, and variations thereof, unless otherwise expressly stated, should be construed as open ended as opposed to limiting. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise.

Furthermore, although items, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further embodiment includes from the one particular value and/or to the other particular value. The recitation of numerical ranges by endpoints includes all numbers and fractions subsumed within the respective ranges, as well as the recited endpoints. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosure. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “optional” or “optionally” means that the subsequent described event, circumstance or substituent may or may not occur, and that the description includes instances where the event or circumstance occurs and instances where it does not.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present disclosure. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the disclosure. For example, in the appended claims, any of the claimed embodiments can be used in any combination.

All patents, patent applications, published applications, and publications, databases, websites and other published materials cited herein are hereby incorporated by reference to the same extent as though each individual publication, published patent document, or patent application was specifically and individually indicated as being incorporated by reference.

The current disclosure will give healthcare systems, providers, and educational institutions distinct advantages by providing patients and learners with a novel approach to medical care and education. It also has the potential to improve the quality of care and satisfaction of patients, providers, and learners. The projection system and software would also provide a competitive advantage in the medical imaging and diagnostic device industry as compared to existing technology and diagnostic methods.

Morphing is a special effect in motion pictures and animations that changes (or morphs) one image or shape into another through a seamless transition. Traditionally, such a depiction would be achieved through dissolving techniques on film. Since the early 1990s, this has been replaced by computer software to create more realistic transitions. A similar method is applied to audio recordings in similar fashion, for example, by changing voices or vocal lines.

In the early 1990s, computer techniques that often produced more convincing results began to be widely used. These involved distorting one image at the same time that it faded into another through marking corresponding points and vectors on the “before” and “after” images used in the morph. For example, one would morph one face into another by marking key points on the first face, such as the contour of the nose or location of an eye, and mark where these same points existed on the second face. The computer would then distort the first face to have the shape of the second face at the same time that it faded the two faces. To compute the transformation of image coordinates required for the distortion, the algorithm of Beier and Neely can be used.

Morphing algorithms continue to advance and programs can automatically morph images that correspond closely enough with relatively little instruction from the user. This has led to the use of morphing techniques to create convincing slow-motion effects where none existed in the original film or video footage by morphing between each individual frame using optical flow technology. Morphing has also appeared as a transition technique between one scene and another in television shows, even if the contents of the two images are entirely unrelated. The algorithm in this case attempts to find corresponding points between the images and distort one into the other as they crossfade.

The current disclosure may use morphing to display visual changes of a patient's disease starting with a real-time or recorded visual assessment of the disease state and then use morphing algorithms and other forms of artificial intelligence (AI) and deep learning (DL) to visually project progression, regression, or no change of disease over time. Morphing applications are becoming commonplace in various industries. Hollywood film makers use novel morphing technologies to generate special effects. Indeed, Disney uses morphing to speed up the production of cartoons. Among so many morphing applications, the current disclosure is specifically interested in disease condition morphing as this would allow comparison of a disease in a patient over time by comparing separate instances of visual/patient data to generate a morphed disease condition. Indeed, prognosis generations showing future disease condition evolution/progression are also capable. Further, individual patient data may be compared to patient's lacking the disease condition to show how the patient's medical condition has evolved from a status of “healthy” or no medical condition to the patient's current medical status and indeed beyond to projections/estimates of future medical status based on the patient's medical data/input. Suitable morphing technologies would be able to identify particular locations in or on the human body and capture changes in same while also being able to show the progress from an earlier captured image to a later captured image by “morphing” the prior image into the later image to show the change in medical condition, whether an improvement or decline in the medical condition. This may include feature finding, image partitioning, coordinate transformations, and/or cross-dissolving as known to those of skill in the art. See, e.g., Jonas Gomes et al. “Warping and morphing of graphical objects”, Morgan Kaufmann Publishers (1999) and Morphing, CESCG Seminar 1997 by Thomas PeNKLeR. Examples of potential morphing platforms include but are not limited to: After Effects, Elastic Reality, FantaMorph, Gryphon Software Morph, Morpheus, MorphThing, Nuke, SilhouetteFX, and/or FotoMorph.

The real-time visual assessment of a disease state can include any visual assessment used in medicine such as ultrasound for heart failure, fundoscopic eye examination for diabetic retinopathy, camera captured skin lesions for psoriasis, biopsy histology specimens for kidney disease, or any other form of visual assessment of a medical condition used today or developed in the future by those of skill in the art. The visual assessment can include the full spectrum of visual formats from still images (skin lesion) to 3D video (heart ultrasound) and include a full range of image resolutions.

The exact morphing technology used will be dependent on the disease entity and the complexity of the medical image of interest with respect to features such as shape, size, geometric components, number and location of important morphing contact points, and color spectrum. Other important consideration include whether the bodily tissue is still (e.g., skin or brain) or dynamic (e.g., heart or lungs) and the desired resolution of specific components of the morphed image that would be best for the intended use as they may differ for patient education, health care learner education, clinical decision making, or a specific medical research endeavor.

Thus, primary processes in morphing such as coupling of warping (geometric transformation) of the image and color interpolation, and cross-dissolving can be approached from a number of different technical perspectives and will be determined by specific image properties, disease processes, and intended use needs. In addition, the success of effective morphing program development also depends on the quality and quantity of relevant image availability. In general, a good outcome will require some degree of experimentation to determine the ideal morphing process and an appropriately large and high-quality image source of the disease process for the primary intent of the morphed images or video. Some disease processes can be well projected with a series of intermediate still images but others are best projected with short videos, especially those that include dynamic structures and/or have simultaneous visual pathological processes ongoing.

On the most basic level, a patient's image can be used as the source image and a standard visual of the disease process of interest as the target image. A combination of commercially available morphing software can be used to create a series of intermediate morphed images and/or video to be used for general education purposes. A more advanced version suitable for healthcare provider education, clinical decision making, and research would need to include the development of AI morphing software based on an appropriately large patient population and image database that could be used for more precise and personalized projection of disease progression, regression, or stability.

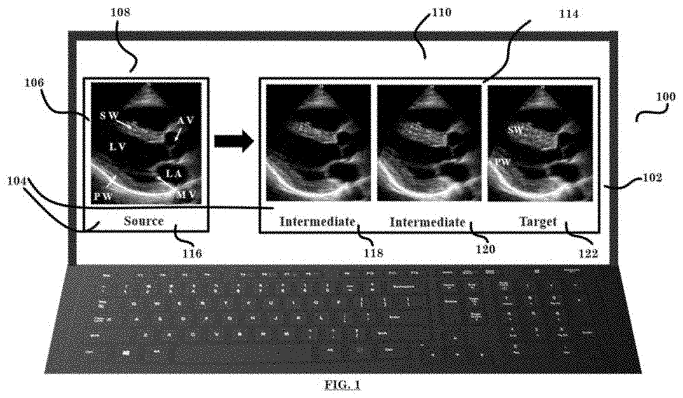

Referring to FIG. 1, a system 100 of the current disclosure will include a viewing screen 102 with split-screen capability 104 in which the patient's visual assessment 106 (still image, video loop, etc.) will be displayed and independently controlled on first side 108 of viewing screen 102 and the AI/DL analyzed and driven visual morphing projection 114 will be displayed on second side 110 for comparison. Viewing screen 102 may be a laptop 112, or other suitable device such as a desktop, imaging system, medical device, smart pad or tablet, smart phone, etc., capable of receiving and displaying patient's visual assessment 106 and visual morphing projection 114, as known to those of skill in the art.

FIG. 1 shows one embodiment a portable version of the image and video viewing system with a hypertensive patient's echocardiogram on first side 108 followed by morphed projected images on second side 110 of viewing screen 102. The important heart structures are labeled: left ventricle (LV), septal wall (SW), posterior wall (PW), left atrium (LA), aortic valve (AV), and the mitral valve (MV). Note the projected thickening of the septal and posterior walls of the left ventricle from source image 116 of the patient through first intermediate image 118 and second intermediate image 120 to projected target image 122 of left ventricular hypertrophy resulting from chronic hypertension. While only a single source image 116 is shown, along with two intermediate stages 118 and 120, and a single projected target image 122 and project, the current disclosure is not so limited and more source images, intermediate stages and projected target images are considered disclosed within the scope of this device. Multiple source images, as well as more intermediate and target images, may be displayed for evaluation and projection on viewing screen 102. Thus, in one embodiment, FIG. 1 shows one embodiment of a Projection System for Visual Morphing of Real Time or Recorded Patient Disease with a patient's real time image of the heart on the left and the projected image on the right with increased thickness of the muscular wall due to uncontrolled high blood pressure at one year.

The display techniques of the current disclosure are varied. For instance, disease projection can be displayed as a set of still images at various time intervals (e.g., 1 week, 1 month, 1 year or intermediate time points within these intervals) or can be displayed as a continuous video that can be advanced at variable speed (e.g., 1 month per second or every 5 seconds) or a specific time sequence can be selected (e.g., projection for 6 months). The variable speed of projection or rate of transition can be based on the number and extent of known risk factors that can accelerate disease progression. The entire screen including the real-time/recorded patient image and the AI projected images can be captured as an image or video and archived for future review or development of educational material and made available to the patient and family members to enhance understanding of the disease process.

The software to be used with the current disclosure may be thought of as a unique combination of the type of artificial intelligence and deep learning being used in medicine for diagnosis and treatment purposes, software for medical image interpretation, and the age-progression/morphing animation software being used in areas such as forensics to project how individuals would look years later based on earlier pictures of them. See, Kemelmacher-Shlizerman I, Suwajanakorn S, Seitz S M. Illumination-Aware Age Progression. Proceedings of the IEEE Conference on computer vision and pattern recognition. 2014: pages 3334-3341. https://openaccess.thecvf.com/content_cvpr_2014/papers/Kemelmacher-Shlizerman_Illumination-Aware_Age_Progression_2014_CVPR_paper.pdf.

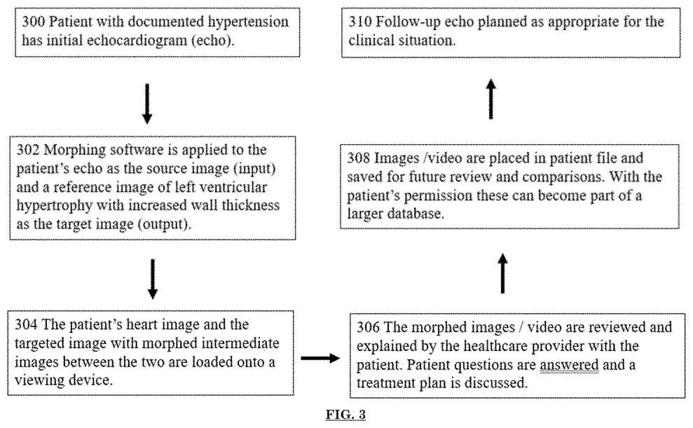

The basic version protocol of one embodiment of a system/method of the current disclosure can be seen in FIG. 3, which shows a method for applying morphing software to illustrate medical condition projections for medical disorders. At step 300, a patient with documented hypertension has an initial echocardiogram (echo) with imaging of the left ventricle of the heart for assessment of ventricular wall thickness that will serve as the source image (input) for morphing to project the effects of hypertension over time. At step 302, morphing software is applied to the patient's echo as the source image (input) and a standard reference image example of heart changes of left ventricular hypertrophy with increased wall thickness serves as the target image (output). At 304, the patient's heart image is displayed side-by-side with the targeted image with morphed intermediate images between the two images. These can be a series of still images or a morphing video can be created from the source to the target image. At step 306, the morphed images or video are reviewed by the healthcare provider with the patient and the effect of chronic hypertension on the heart is explained. Patient questions can be answered and a treatment plan can be discussed to include how best to prevent progression of the disease and possibly reverse the pathological changes of the disease. The importance of medication, regular blood pressure monitoring, and life style changed can be included in the discussion. At step 308, the images and/or video are placed in the patient's file and saved for future review and comparisons. Over time with additional echocardiograms, a personalized lifetime visual history of the patient's cardiac response to hypertension and treatment regimens will be created. This series of archived images and/or videos can also, with the patient's permission, become part of a valuable large database for additional AI training, education, and research on the topic. At step 310, a follow-up echocardiogram can be planned as appropriate for the clinical situation.

An alternate embodiment of the system/method may use AI/DL morphing algorithms developed on big data of known populations with and without the disease of the patient. The morphed projection would be “personalized” and more “precise” to the specifics of the patient by matching the patient's profile to factors known or those determined by AI training to influence the disease process and its visual characteristics over time. These factors could include but would not be limited to demographics such as age, sex, race, nationality; clinical data such as height, weight, change in weight, blood pressure readings; imaging data such as x-rays, ultrasound, MRI, and CT; laboratory data such as hemoglobin, blood sugar, blood chemistries; and other important health related data such as concurrent diseases (e.g., coronary artery disease, diabetes), medications, allergies, life style characteristics (e.g., smoking, alcohol consumption, exercise activity, sun exposure), and the patient's genetic profile.

Factors used to accurately morph a patient's disease progression visually would be determined by the AI/DL analysis specific for the disease entity of interest (i.e. heart diseases, eye diseases, skin diseases) and the corresponding visual changes of disease pathology from the databases of the disease relevant patient populations. The degree of visual morphing and rate of morphing will be based on the number and extent of known risk factors that can accelerate disease progression. These will be identified and weighted by AI training on the appropriate patient populations and applied to the individual patient based on their documented risk factors.

For example, in the case of development of left ventricular hypertrophy and heart failure from hypertension, relevant data to consider based on established national and international guidelines and recommendations that affect blood pressure include age, sex, race, extent and duration of hypertension, body mass index (BMI), smoking history, and regular activity level among several other factors. See, Centers for Disease Control and Prevention. High Blood Pressure Portal: https://www.cdc.gov/bloodpressure/index.htm. Development of AI software including algorithms, deep learning, and convoluted neural networks would adhere to well-established developmental and research protocols for AI and literature review guidelines including such essential features as proper selection of patient databases, appropriate mathematical operations/logistic regressions, adherence to rigorous protocol training decisions (i.e. supervised versus unsupervised training), testing (i.e. appropriate data and independence of the testing datasets from training sets), avoidance of overfitting, and validation of the projection software (i.e. independent and appropriate populations with meaningful and objective outcome measures) to ensure valid patient projection for the patient population, disease entity, and visual features utilized. See, Liu Y, Chen P C, Krause J, Peng L. How to Read Articles That Use Machine Learning: Users' Guides to the Medical Literature. JAMA. 2019 Nov. 12; 322(18):1806-1816.

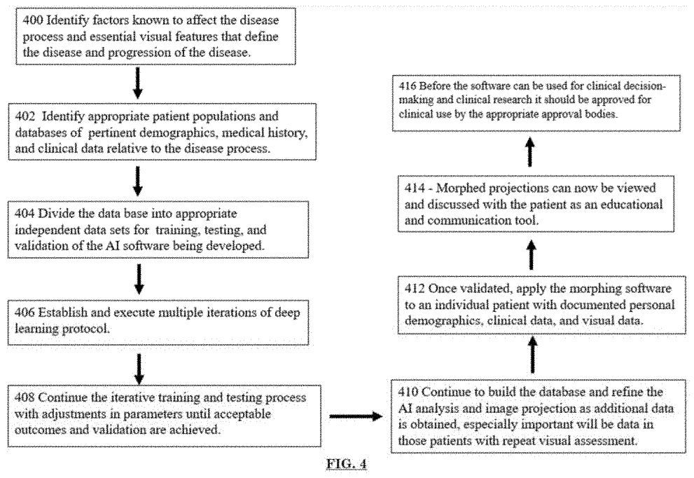

One approach of the current disclosure is summarized in FIG. 4. At step 400, one identifies factors known to affect the disease process of interest and essential visual features that define the disease and progression of the disease from the literature and expert opinion (ground truths). At step 402, one identifies appropriate patient populations for development of AI driven morphing and obtain/create databases of pertinent demographics, medical history, and clinical data including all visual data relative to the disease process. At step 404, one divides the database into appropriate independent data sets for training, testing, and validation of the AI software being developed such as rules-based algorithm, deep learning with supervised or unsupervised training, or a convolutional neural network. At step 406, one establishes and executes multiple iterations of deep learning protocols. At step 408, one continues iterative training and testing process with adjustments in parameters until acceptable outcomes and validation are achieved for the patient populations of interest. At 410, one continues to build the database and refine the AI analysis and image projection as additional data is obtained, especially important will be data in those patients with repeat visual assessments over time. Validation should be performed on a dataset that is independent from training and testing datasets and repeat validation should be performed after significant changes are made in the morphing software. At step 412, once found to be well validated and accurate for visual projection, apply the morphing software to an individual patient with documented personal demographics, clinical data, and visual data to allow accurate morphed projection of the visual image of their disease. At step 414, morphed projections can now be viewed and discussed with the patient as an educational and communication tool. They can also be used to develop additional learning material for patients and health care providers in training and practice. At step 416, before the software can be used for clinical decision-making and clinical research it should be approved for clinical use by the appropriate approval bodies such as the Food and Drug Administration (FDA) and the Institutional Review Board (IRB).

This approach allows unique personalized patient projections of visual disease changes based on their individual demographic and medical data profile. This level of precision would then allow the projection to not only be used for education but also to assist in medical decision-making, track medical outcomes, and used as a research tool. In addition, the creation of morphed images by the software could be considered for use in extending or augmenting datasets for other AI image dependent training.

Projection of the natural progression of the disease without treatment, one of the important patient populations for AI training, can be developed from patient populations not treated for a variety of reasons such as poor access to follow-up treatment, limited resources to obtain medications, and non-compliance with treatment regimens. Projection of disease progression, remission, or no change with various treatments regimens will be developed from treatment populations and used to more accurately project the individual patient's disease course. Over time, using AI and DL with large databases, additional factors important in projecting the disease course of individuals will likely be identified with unsupervised AI training and incorporated into the Projection System.

The AI/DL analytic and morphing software may be built into the viewing system or can be downloaded for use on a desktop, laptop, tablet, smart phone, or other compatible viewing devices. Alternatively, the software can be downloaded onto a medical imaging system that is compatible and has adequate viewing capability. This projection capability would allow immediate real-time analysis and projection for clinical decision-making and education as well as increase the commercial value of imaging systems such as ultrasound machines.

To better appreciate the need and use of such a projection system, the prevalence of hypertension and the example of chronic hypertension leading to heart remodeling and the development of heart failure can be further explored. Hypertension is a worldwide health issue affecting more than one billion individuals and causing an estimated 9.45 million deaths per year. In the United States, high blood pressure was considered a primary or contributing cause in more than 360,000 deaths in 2013. Hypertension is not only a major contributor to coronary artery disease, the pressure load on the heart from the hypertension contributes directly to thickening of the heart muscle itself that is a compensatory mechanism to address the increased pressure load it must pump against.

However, if the high blood pressure persists, despite adequate short-term compensation, the heart can eventually fail and not be able to pump enough blood to keep up with the body's needs. The failing heart will enlarge, the walls will become thin, and the amount of blood pumped with each heartbeat will fall dramatically. Heart ultrasound or echocardiography (echo) can visually document all of these findings from the progressive incremental increase in left ventricle wall thickness to the enlarging heart and progressive decrease in ejection fraction (the percentage of blood ejected from the heart with each heart beat) of the failing heart.

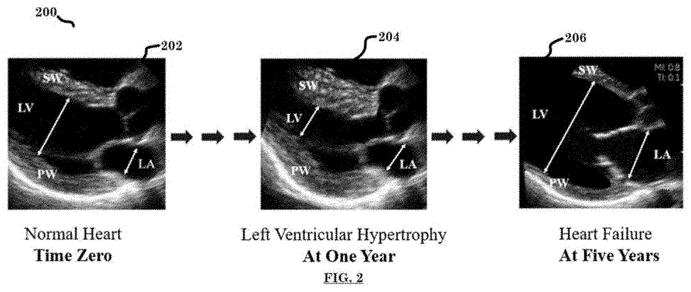

Thus, a patient diagnosed with hypertension can undergo an echocardiogram and the system's software using the patient's own echo will project the course of the effect of uncontrolled hypertension on the heart for side-by-side for comparison and discussion, see FIG. 2. FIG. 2 shows a system projection 200 for a patient's echocardiogram at time zero 202 to ventricular wall thickening and enlarged left atrium at one year 204 and heart failure at five years 206. A follow-up echocardiogram can be compared to the original echo and where along the continuum from normal heart to heart failure it fits in the medical condition progression/evolution. Improvement in the condition (regression of disease) can be noted, if present, and the patient congratulated on the improvement and encouraged to continue working on maintaining a healthy and safe blood pressure.

If a patient already has visual evidence of disease on the original echo, the system can create a retrospective image of what their normal heart would have looked like prior to the disease process affecting it. This image can also become a therapeutic goal with efforts to return to the normal healthy state of the heart. Additional data such as measurements of the thickness of the muscular wall of the heart and the ejection fraction of each beat of the heart can be determined by most ultrasound machines and used to further support and reinforce the changes seen visually by the patient and the healthcare provider.

Another application of the current disclosure would be to use morphing software to map out interval changes between diagnostic imaging studies. For example, with respect to patients who have serial echocardiograms, the system would create a visual representation between two or even three studies so the patient can better appreciate changes taking place between the imaging studies such as visually showing a decrease in ventricular wall contraction and a corresponding decrease in the ejection fraction or percent of blood ejected during the cardiac cycle as the heart failure worsens decrease over time. This application of the system would represent a clinical use for healthcare providers to visually track changes in diagnostic studies apart from its ability to project a future state.

With this application the focus is not on projecting to what a future, generic image might look like but instead uses the patient's earlier image as the morphing source (input) and the new image of the patient as the target (output) and shows how the heart has morphed from the image obtained during the previous echocardiography imaging session to the present session. This would result in a personalized and accurately morphed image process that could also be used for education, research, and clinical decision-making.

Unfortunately, patients are often unable to truly understand their health or a disease condition, how it can progress, and why they need to take medications and change certain aspects of their lifestyles. These are sometimes referred to as issues of health literacy and can lead to problems in getting patients engaged in their own care and being compliant with the prescribed medical plan. In the management of hypertension, compliance can be a particularly difficult problem because the patient in general does not feel sick until significant damage has been done and they experience symptoms of heart failure such as shortness of breath or they experience a life-threatening event such as a stroke. However, there are studies that suggest that patients who are shown health-related pictures and not just told about them or are presented information about the disease in general are more engaged in their health care, have increased understanding of their condition, and can positively affect the healthcare provider-patient relationship. See, Houts P S, Doak C C, Doak L G, Loscalzo M J. The role of pictures in improving Health Communication: a review of research on attention, comprehension, recall, and adherence. Patient Educ. Couns. 2006; 61(2):173-190, and Phelps E M, Wellings R, Griffiths F, Hutchinson C, Kunar M. and Do medical images aid understanding and recall of medical information? An experimental study comparing the experience of viewing no image, a 2D medical image and a 3D medical image alongside a diagnosis. Patient Educ Couns. 2017; 100(6):1120-1127, and Carlin L E, Smith H E, Henwood F. as well as To see or not to see: a qualitative interview study of patients' views on their own diagnostic images. BMJ Open. 2014; 4:e004999. This could lead to better compliance with their treatment plan and better medical outcomes.

Because of the many variables involved in the course of a patient's disease (state of the disease at the time of diagnosis, age, sex, concurrent health problems, life style, genetic makeup, etc.) and the interaction of these variables, it is very difficult for healthcare providers to process the large volume of all this data and make confident predictions. Many practitioners are reluctant to do so and patients can feel lost in not knowing what to expect and feel like they have little control over their disease. However, with AI, DL, and big data, medical diagnoses and more accurate predictions can be made. Adding a visual representation to these predictions can create a powerful means of communication and an important patient education tool that ultimately adds significant value to the medical experience and can improve patient outcomes.

In addition to direct patient care, this visual projection system based on AI/DL and big data offers a unique teaching tool for healthcare providers in training to better understand the pathophysiology of disease processes, clinical assessment, and disease management. The current disclosure is capable of producing unique learning material for the health professions as well as the public. It can even provide primary, secondary, and college level life science teachers new ways to teach their subject matter.

The visual data from the current disclosure also has the potential to add a unique form of disease assessment in evaluating treatment regimens, especially new pharmaceuticals. The AI/DL analytics will also be capable of identifying previously unrecognized factors in the disease process and expand our understanding of the pathophysiology of the disease. Thus, the System has potential as a unique research tool. As a research tool in AI development, morphed images have the potential to be used to augment datasets to provide more examples for AI training.

Novel elements of the System include but are not limited to: (1) creation of a visual projection of a patient's disease using the patient's own medical image and databases of patients with factors affecting the disease; (2) creation of a retrospective visual image of the patient's original state of health prior to the disease process; and (3) creation of large, visually oriented databases that can be used for patient care, education, and research.

Referring again to FIG. 2, it shows an example of the system's morphed projection of a patient with hypertensive disease at risk of developing heart failure. The results project from the normal appearance of the heart on the left to marked thickening of the left ventricular walls or left ventricular hypertrophy and then progression to heart failure to the far right. Note the marked projected increase in the volumes of the left ventricle and left atrium and thinning of the ventricular walls at five years. Arrows indicate a series of morphed images from one shown image to the next. The patient is a 52-year old African-American male with newly diagnosed hypertension (160/95) and a normal echocardiogram at time zero. If the hypertension is not brought under control, the disease projection from artificial intelligence and deep learning for this patient at 1 year will show marked thickening of the wall of the left ventricle as the heart has to work against the high blood pressure and compensates by increasing the wall muscle. This has made it harder for the left ventricle to accept blood from the left atrium resulting in an enlarging left atrium. If the high blood pressure continues the echocardiogram at 5 years will show heart failure with marked increase size of the chamber of the left ventricle with less blood being ejected with each heartbeat, the wall is now thinning, and the left atrium is significantly enlarged. The patient at this point will likely have fatigue and shortness of breath with even mild exertion.

The projected cardiac images and clinical scenario could be discussed with the patient so he can better understand what effects high blood pressure can have on his heart if not brought under control. Questions can be answered and a treatment approach discussed. A shared decision could be made as to the best treatment approach for him. His own initial echocardiogram can be used as a baseline for visual assessment of the effectiveness of treatment. Follow up echoes can be used as target images to view the morph changes between echoes. With additional information ever improving predictions can be made on the outcome of hypertension on his heart. This would help reinforce a successful treatment outcome or suggest the need for a treatment change or recommitment to the treatment regimen including life style changes if images begin showing changes of disease progression or unsatisfactory improvement.

The projection of disease progression as represented in FIGS. 1 and 2 are based on the patient's own echocardiogram as the source or input image with morphing algorithms applied to produce a series of morphed images ending in the target or output image which can be a reference image of classic left ventricular hypertrophy or heart failure due to hypertension. The projected images can also be determined by deep learning and convolutional neural networks developed from large databases of patient populations specific for a particular visual disease effect. For this hypertension cardiac example those would be large databases of patient images of the development of left ventricular hypertrophy and heart failure and relevant patient demographics and clinical data, especially those known risk factors in development of left ventricular hypertrophy and heart failure. Such a developed morphing program could then use an individual patient's demographics, clinical data, and risk factors to project a personalized set of morphed images or video. This would allow an even more powerful patient education tool as well as a significant tool for clinical decision making and medical research.

Echocardiology and hypertension have been used as just one example of how using visual assessment and projection of disease states can be used to educate patients, improve the healthcare provider relationship, and healthcare outcomes. Many other examples exist with their own set of visual assessment tools, visual pathology, and associated risk factors. However, the same conceptual framework of projection of health and disease states with visual morphing algorithms, deep learning, and CNN's can be developed, applied across these other disease states and improve healthcare for a wide variety of patients.

Systems and methods of the current disclosure provide unique uses not currently available to patients and medical practitioners. The disclosure provides a unique combination of artificial intelligence, medical data analysis, and morphing technology to project disease visual appearance for education, clinical care, and research. It also provides morphing of a patient's clinical images to enhance the patient-healthcare provider relationship and shared decision-making and can be combined with artificial intelligence analysis to personalize morphed images to enhance patient education and clinical decision making. The use of AI-clinical data driven morphed images can extend and augment image datasets for artificial intelligence training. Artificial intelligence morphed images may be used to assess effectiveness of medical therapeutics. Indeed, the current disclosure allows for incorporation of morphing algorithms and artificial intelligence into medical imaging devices and smart cameras/phones for real-time clinical assessment, education, and clinical decision-making. Aspects of the current disclosure may be incorporated into educational systems and learning portals for visual learning of pathology, pathophysiology, and therapeutics. Indeed, the current disclosure provides for especially useful “rate of morphing” analysis for disease progression correlated and matched to number and extent of known risk factors that accelerate disease progression. This disclosure may also enable artificial intelligence clinical data-driven morphed projection to assess treatment options. Indeed, capture of patient disease morphing images and/or video can lead to creation of learning material for healthcare professional and others. One particular useful aspect of the current disclosure is the capability for “backward morphing” of a patient's images from pathological based on the patient's existing medical status to normal healthy images for enhanced understanding and establishment of a visual goal of treatment and to encourage patient compliance with treatment regimes. Further, use of images from previous image examinations may serve as source images (morphing input) to follow-up exam images as target image (output) to accurately morph changes from one exam to the next for education, research, and clinical decision making. Frequent image acquisition and artificial intelligence driven morphing can also be used to better define the pathological processes and roles of risk factors both established and newly identified by the system and methods disclosed herein. Indeed, the system may serve a useful function by showing that a medical status or condition has not change significantly between first and second images to provide information on treatment regimes, medical prognosis or disease state.

Various modifications and variations of the described methods, pharmaceutical compositions, and kits of the disclosure will be apparent to those skilled in the art without departing from the scope and spirit of the disclosure. Although the disclosure has been described in connection with specific embodiments, it will be understood that it is capable of further modifications and that the disclosure as claimed should not be unduly limited to such specific embodiments. Indeed, various modifications of the described modes for carrying out the disclosure that are obvious to those skilled in the art are intended to be within the scope of the disclosure. This application is intended to cover any variations, uses, or adaptations of the disclosure following, in general, the principles of the disclosure and including such departures from the present disclosure come within known customary practice within the art to which the disclosure pertains and may be applied to the essential features herein before set forth.

What is claimed is:

1. A method for generating and projecting disease visual appearance comprising:
providing a morphing software platform;
inputting at least one set of medical data into the morphing platform;
generating at least one visual interpretation of a change in medical status from the morphing platform based on the at least one set of medical data.

2. The method of claim 1 further comprising, analyzing the at least one set of medical data with an artificial intelligence platform to assist with generating the visual interpretation of the change in medical status.

3. The method of claim 1 further comprising, projecting a disease visual appearance formed from the visual interpretation of the change in medical status.

4. The method of claim 1 further including employing the method in an educational, clinical care, and/or research setting.

5. The method of claim 1 further comprising, displaying the visual interpretation of the change in medical status in a patient-healthcare provider relationship.

6. The method of claim 1 further comprising, assessing impact of a medical treatment via comparison of the visual interpretation of the change in medical status over a course of the medical treatment.

7. The method of claim 1 further comprising, augmenting at least one image dataset for artificial intelligence training based on analysis of the visual interpretation of the change in medical status.

8. The method of claim 1 further comprising, designating at least one generated first visual interpretation of a first change in medical status from a first image examination as a source image morphing input and designating at least one generated second visual interpretation of a second change in medical status from a second image examination as a target image output and forming a morphed visual interpretation via comparing the first visual interpretation and the second visual interpretation to show changes in a medical condition.

9. The method of claim 8, wherein the at least one generated first visual interpretation is taken earlier in time as compared to the at least one generated second visual interpretation.

10. The method of claim 8, wherein the at least one generated first visual interpretation is taken later in time as compared to the at least one generated second visual interpretation.

11. The method of claim 1 further comprising, generating a backward morphing of patient medical condition images via comparing the visual interpretation of the change in medical status from the morphing software platform based on the at least one set of medical data to at least one image where no medical condition is present.

12. The method of claim 1 further comprising, defining a pathological processes via comparing at least two distinct visual interpretations of the change in medical status.

13. The method of claim 1 further comprising, capturing patient disease morphing images and/or video.

14. The method of claim 1 further comprising, using the at least one visual interpretation to assess treatment options for a medical condition.

15. The method of claim 1 further comprising, assessing disease progress via analysis of the at least one visual interpretation.

16. The method of claim 1 further comprising, incorporating morphing algorithms and artificial intelligence into medical imaging devices for real-time assessment of a medical condition.

17. A system for projecting disease visual appearance comprising:
an artificial intelligence platform;
at least one set of medical data;
a morphing software platform;
wherein the artificial intelligence platform works in tandem with the morphing software platform to show a visual change in a medical status based on the at least one set of medical data.

18. The system of claim 17 further comprising, a display for showing a disease visual appearance formed from the visual interpretation of the change in medical status.

19. The system of claim 17 further comprising, employing the system in an educational, clinical care, and/or research setting.

20. The system of claim 17 further comprising, a morphed visual interpretation created from at least one generated first visual interpretation of a first change in medical status from a first image examination as a source image morphing input and at least one generated second visual interpretation of a second change in medical status from a second image examination as a target image output to show changes in a medical condition.
